(19) United States
(12) Patent Application Publication
(10) Pub. No.: US 2013/0041397 A1
(43) Pub. Date: Feb. 14, 2013

(54) ACUPUNCTURE TOOL

(71) Applicant: Junichi Nishimura, Osaka (JP)

(72) Inventor: Junichi Nishimura, Osaka (JP)

(73) Assignee: TRUSTLEX CO., LTD., Osaka (JP)

(21) Appl. No.: 13/571,561

(22) Filed: Aug. 10, 2012

(30) Foreign Application Priority Data
Aug. 10, 2011 (JP) 2011-174571

(51) Int. Cl. A61B 17/34

(52) U.S. Cl. 606/189

(57) ABSTRACT

An acupuncture tool for performing acupuncture includes an injection needle and an implant thread. The implant thread has one end side being inserted into a pore at a tip side of the injection needle and another end side being drawn so that when the injection needle is inserted into a body, the implant thread is also simultaneously implanted in the body and the injection needle is then extracted with the implant thread left in the body. The injection needle has a predetermined fastener movably mounted thereon. The fastener holds the other end side of the implant thread along the injection needle, and is configured to move in a direction reverse to a direction of inserting the injection needle while the fastener is holding the implant thread during a period from when the injection needle is inserted into the body to when the implant thread is implanted in the body.

### TECHNICAL FIELD OF THE INVENTION

The present invention relates to an acupuncture tool for performing acupuncture to treat a disease, promote health, and carry out other purposes by stimulating a body with the use of the acupuncture tool.

### BACKGROUND OF THE INVENTION

In recent years, various operations have been used in reconstructive and cosmetic surgery treatments as methods of removing wrinkles and reducing (lifting) the sag of the cheeks or others. For example, as disclosed in Japanese Unexamined Patent Application Publication Nos . 2011-136153 and 2008-228993, there is an operation of injecting a beauty essence such as a hyaluronic acid, a Botox silicon, or others by an injector. In this operation, the beauty essence or the like is injected into the inside of the skin at a portion where winkles are to be removed or a portion to be lifted. This operation is simple without incision and immediately effective, and is therefore much in demand. However, this injecting operation has a short effect-lasting period and does not reproduce a skin tissue, and therefore injection or administration is continuously (frequently) required, disadvantageously imposing a physical and mental burden and also increasing cost. Above all, in the conventional injecting operation into the body including a Botox injection, many side effects occur to the body, such as facial paralysis, a change in facial features, and pressuring a respiratory system to possible death.

To address these problems, as a reconstructive and cosmetic surgery treatment with less side effects on the body, there is an operation as disclosed in Japanese Unexamined Patent Application Publication No. 2005-304671 in which a gold (Au) thread or a platinum (Pt) thread are implanted in the body. In this operation, in a needle for surgery for use in suture operation or the like with one end made acute and another end, a gold thread or a platinum thread is inserted into the other end and is implanted so as to suture the inside of the skin. With the physiological effect included in gold and platinum, skin cells are activated to stop the progress of aging, thereby reducing wrinkles, blotches, sag, and others of the skin.

However, the above-described constructive and cosmetic surgery treatment of implanting a gold thread or a platinum thread with a needle for surgery has a problem in which this treatment cannot be applied to patients having an allergy to noble metal prevailing in recent years. Also, since the breadth and depth of the skin that can be sutured has a limitation, implantation can be performed on not everywhere. Moreover, in a treatment in which the needle for surgery is inserted twice in one suture into a plurality of places and the gold thread or the platinum thread is then cut out, the treatment requires a long time and causes a lot of fatigue on the body. In addition, the gold thread and the platinum thread are made of expensive metals, requiring large cost. Still further, the gold thread or the platinum thread is inserted into a concave formed at the other end of the needle for surgery and is then crimped from its outer surface. Therefore, the needle body is deformed and cannot be reused. Still further, if a metal thread such as a gold thread or a platinum thread is implanted in the body, a glare of the implanted gold thread or platinum thread appears when an X ray or a CT scan is taken for a checkup, thereby causing an inconvenience in the checkup. This problem is particularly serious for the head part including the face.

### SUMMARY OF THE INVENTION

Meanwhile, an operation with an acupuncture treatment for use in orthopedic medical cares and various fields has been performed in reconstructive and cosmetic surgery treatments. Acupuncture is a medicine methodology in search of beauty hidden inside a person and inside his or her body. Due to aging without an external wound, skeletal or muscular problems inside and outside of the body, and other reasons, a disorder occurs in the course of aeremia, which is a flow of body energy, and an abnormality occurring to the outer shape due to this disorder can be sufficiently returned to the original position through acupuncture treatment. In acupuncture, the body can be balanced, the circulation of the blood can be improved, unbalanced growth can be alleviated, and a beautiful body with ideal muscles and skeleton can be recovered. Specifically, through a needle, not only a relevant region of the body but also other related acupuncture points, meridians, and acupuncture muscles also undergo operation. With this, many effects can be achieved, such as an improvement in the circulation of the blood, removal of muscular fatigue, and balancing the inner body. In the reconstructive and cosmetic surgery treatments, it is said that effects can be achieved such as stimulating the inside of the skin for activation, stopping the progress of aging, and improving the s kin for renewal.

However, while this acupuncture treatment has been applied in reconstructive and cosmetic surgery, this is not immediately effective, much effect cannot be expected, and a significant effect cannot be obtained. Moreover, there is a problem in which its effect greatly depends on the capability of the acupuncturist. That is, while the acupuncture treatment can be applied in reconstructive and cosmetic surgery, a tool for use in this treatment itself is similar to that for traditional acupuncture treatment, and therefore its utilization range cannot be widened under the actual circumstances.

Thus, an object of the present invention is to provide an acupuncture tool for performing acupuncture, the tool capable of further stimulating the body and also being sufficiently utilized for reconstructive and cosmetic surgery.

The acupuncture tool for performing acupuncture of the present invention of the application to treat a disease, promote health, and carry out other purposes by stimulating a body, and the acupuncture tool includes an injection needle and an implant thread to be implanted in the body. The implant thread has one end side being inserted into a pore at a tip side of the injection needle and another end side being drawn so that when the injection needle is inserted into an affected area of the body, the implant thread is also simultaneously implanted in the body and the injection needle is then extracted with the implant thread left in the body. The injection needle has a predetermined fastener movably mounted thereon, the fastener holds the other end side of the implant thread along the injection needle, and is configured to move in a direction reverse to a direction of inserting the injection needle while the fastener is holding the implant thread during a period from a time when the injection needle is inserted into the affected area of the body to a time when the implant thread is implanted in the body.

According to the present invention, from the time when the injection needle is inserted into the affected area of the body to the time when the implant thread is implanted in the body, the fastener moves along the injection needle while holding the implant thread. Therefore, even a relatively long implant thread can be easily implanted.

According to the present invention, when the injection needle is inserted into the affected area of the body, the implant thread is also simultaneously implanted in the body. Therefore, the implant thread can be implanted in the skin and the body while stimulating the inside of the skin (the body). Then, when the acupuncture tool, that is, the injection needle, is extracted after providing stimulation, the implant thread inserted into the pore at the tip of the injection needle comes off from the injection needle and is left in the body. Therefore, the stimulating effect can be kept long.

The inventor of the present application has found that when an injection needle for surgery and a suture for surgery are used and the suture for surgery is implanted in a predetermined part of the body, the effect in treating a disease, promoting health, and carrying out other purposes can be increased and an effect of smoothing out wrinkles and reducing the sag of the cheeks or others (a lifting effect) can be achieved.

That is, in the present invention, preferably, the injection needle is an injection needle for surgery and the implant thread is a suture for surgery. Also in the present invention, a drawn length of the implant thread to be drawn from the pore of the injection needle is preferably set longer than an insertion length of the implant thread to be inserted into the pore of the injection needle.

According to the present invention, an existing injection needle for surgery and an existing suture for surgery can be used to readily perform acupuncture. Also, the suture for surgery is absorbed into the body and does not adversely affect the body. Also, a chemical solution or pharmaceutical, such as vitamin, collagen, or ointment, having an effect of stimulation, smoothing out wrinkles, or others may be applied to this implant thread for use. Furthermore, the injection needle is an existing needle for surgery and is therefore inexpensive. Still further, if sterilized, the injection needle can be recycled again and again and is therefore economical. When a metal thread such as a gold thread or a platinum thread is implanted in the body, a glare of the implanted gold thread or platinum thread appears when an X ray or a CT scan is taken for a checkup, thereby causing an inconvenience in the checkup. This problem is particularly serious for the head part including the face. By contrast, an existing suture for surgery is used in the present invention, and therefore no inconvenience occurs in the checkup.

Preferably, in the present invention, the fastener is circular along an outer perimeter of the injection needle, and is made by using a cushioning elastic member.

The fastener is preferably made of sponge, rubber, synthetic resin, or others. Since the fastener is not to be implanted in the body, the fastener preferably has a color different from those of the injection needle and the implant thread.

Preferably, in the present invention, a base end side of the injection needle and a resin-made operating unit are integrally provided, and a cap covering the injection needle and the implant thread is configured to removably fit in the operating unit, and the injection needle and the fastener are covered so as not to be in contact with the cap.

According to the present invention, with the cap fitting in, the implant thread inserted into the injection needle is prevented from coming off from the injection needle and, also from a sanitary point of view, the injection needle and the implant thread can be each kept clean. The cap has a simple structure as fitting in the operating unit provided integrally with the injection needle, and can be easily removed in a simple manner.

According to the present invention, from the time when the injection needle is inserted into the affected area of the body to the time when the implant thread is implanted in the body, the fastener moves along the injection needle while holding the implant thread. Therefore, even a relatively long implant thread can be easily inserted.

According to the present invention, when the injection needle is inserted into the affected area of the body, the implant thread is also simultaneously implanted in the body. Therefore, the implant thread can be implanted in the skin and the body while stimulating the inside of the skin (the body). Then, when the acupuncture tool, that is, the injection needle, is extracted after providing stimulation, the implant thread inserted into the pore at the tip of the injection needle comes off from the injection needle and is left in the body. Therefore, the stimulating effect can be kept long.

In particular, when an injection needle for surgery and a suture for surgery are used and the suture for surgery is implanted in a predetermined part of the body, the effect in treating a disease, promoting health, and carrying out other purposes can be increased. Also, an effect of smoothing out wrinkles and reducing the sag of the cheeks or others (a lifting effect) can be achieved, because the suture for surgery is absorbed into the body and does not adversely affect the body.

### BRIEF DESCRIPTIONS OF THE DRAWINGS

FIG. 1 is an external perspective view for describing an acupuncture tool for performing acupuncture according to an embodiment of the present invention;

FIG. 2 is a sectional side view for describing the state in which an implant thread is inserted into a pore at the tip of the injection needle in the acupuncture tool of FIG. 1;

FIG. 3 is an external perspective view for describing the state in which a cap is applied over the acupuncture tool of FIG. 1;

FIG. 4A and FIG. 4B are diagrams for describing the state in which the implant thread is implanted by the injection needle of FIG. 1 in the inside of the skin, FIG. 4A depicting that the tip is inserted into the inside of the skin and FIG. 4B depicting that the tip is extracted from the inside of the skin;

FIG. 5A and FIG. 5B are diagrams of an example of use of the acupuncture tool of FIG. 1, FIG. 5A depicting parts where wrinkles are formed on the face and FIG. 5B depicting the state of inserting the acupuncture tool;

FIG. 6A to FIG. 6C are diagrams for describing the progress of effects of the acupuncture tool of FIG. 1 on wrinkles on the forehead, FIG. 6A depicting the state before the use of the acupuncture tool, FIG. 6B depicting a first stage after the use of the acupuncture tool, and FIG. 6C depicting the final stage after the use of the acupuncture tool;

FIG. 7A and FIG. 7B are diagrams for describing effects of the acupuncture tool of FIG. 1 on wrinkles at the corners of the eyes, FIG. 7A depicting the state before the use of the acupuncture tool and FIG. 7B depicting the state after the use of the acupuncture tool;

FIG. 8A and FIG. 8B are diagrams for describing effects of the acupuncture tool of FIG. 1 on wrinkles at the corners of the mouth, FIG. 8A depicting the state before the use of the acupuncture tool and FIG. 8B depicting the state after the use of the acupuncture tool;

FIG. 9A and FIG. 9B are diagrams for describing effects of the acupuncture tool of FIG. 1 on wrinkles on the forehead, FIG. 9A depicting the state before the use of the acupuncture tool and FIG. 9B depicting the state after the use of the acupuncture tool;

FIG. 10A and FIG. 10B are diagrams for describing effects of the acupuncture tool of FIG. 1 on surgery for swelling the forehead, FIG. 10A depicting the state before the use of the acupuncture tool and FIG. 10B depicting the state after the use of the acupuncture tool;

FIG. 11A and FIG. 11B are diagrams for describing effects of the acupuncture tool of FIG. 1 on surgery for straightening the nasal muscle, FIG. 11A depicting the state before the use of the acupuncture tool and FIG. 11B depicting the state after the use of the acupuncture tool;

FIG. 12A and FIG. 12B are diagrams for describing effects of the acupuncture tool of FIG. 1 on surgery for lifting the buttocks, FIG. 12A depicting the state before the use of the acupuncture tool and FIG. 12B depicting the state after the use of the acupuncture tool; and

FIG. 13A and FIG. 13B are diagrams for describing effects of the acupuncture tool of FIG. 1 in a treatment for increasing hairs, FIG. 13A depicting the state before the use of the acupuncture tool and FIG. 13B depicting the state after the use of the acupuncture tool.

### DESCRIPTIONS OF THE PREFERRED EMBODIMENTS

Embodiments for carrying out the present invention are described below with reference to the drawings.

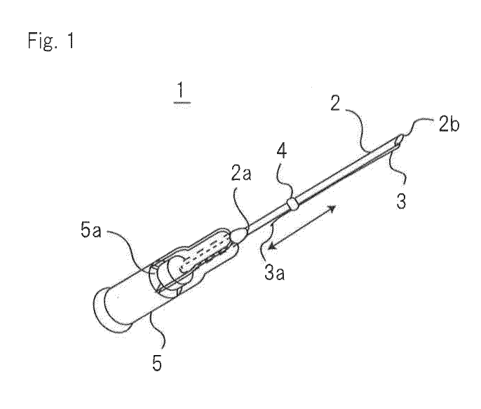

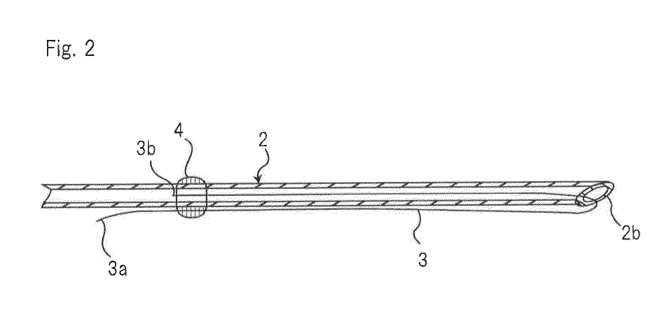

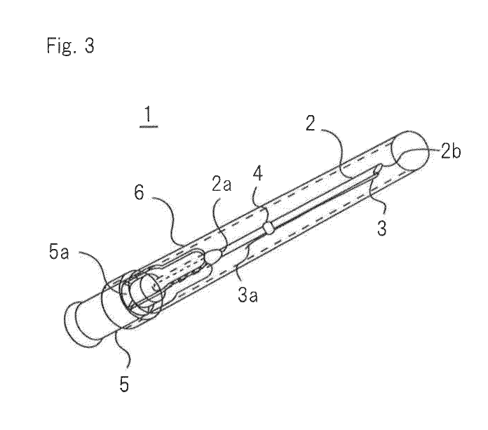

FIG. 1 is an external perspective view for describing an acupuncture tool for performing acupuncture according to an embodiment of the present invention. FIG. 2 is a sectional side view for describing the state in which an implant thread is inserted into a pore at the tip of the injection needle in the acupuncture tool of FIG. 1. FIG. 3 is an external perspective view for describing the state in which a cap is applied over the acupuncture tool of FIG. 1.

An acupuncture tool 1 for performing acupuncture according to the present embodiment is used to treat a disease, promote health, and carry out other purposes by stimulating the body. The acupuncture tool 1 includes an injection needle 2, an implant thread 3 to be implanted in the body, a fastener 4 holding the implant thread 3 along the injection needle 2, and a resin-made operating unit 5 integrally provided to the injection needle 2.

The injection needle 2 has a pore 2b formed at a tip side for use in injecting and suctioning a liquid or air, and an operating unit 5 is integrally provided on a base end 2a side. Here, the injection needle 2 for use in the present embodiment is an injection needle for surgery generally for use, and those having a plurality of thicknesses (an outer diameter of approximately 1.20 mm to 0.40 mm and an inner diameter of approximately 0.40 mm to 0.22 mm) are prepared and used according to the portion for use. For example, any one of injection needles having thicknesses represented by G (gauges) from 18 G to 27 G, which are thickness standards, is selectively used according to the portion for use. The gauges are as follows: 18 G (an outer diameter of 1.20 mm and an inner diameter of 0.40 mm), 19 G (an outer diameter of 1.10 mm and an inner diameter of 0.78 mm), 20 G (an outer diameter of 0.90 mm and an inner diameter of 0.66 mm), 21 G (an outer diameter of 0.80 mm and an inner diameter of 0.57 mm), 22 G (an outer diameter of 0.70 mm and an inner diameter of 0.48 mm), 23 G (an outer diameter of 0.65 mm and an inner diameter of 0.40 mm), 24 G (an outer diameter of 0.55 mm and an inner diameter of 0.37 mm), 25 G (an outer diameter of 0.50 mm and an inner diameter of 0.37 mm), 26 G (an outer diameter of 0.45 mm and an inner diameter of 0.27 mm), and 27 G (an outer diameter of 0.40 mm and an inner diameter of 0.22 mm). Often used is a fine needle (any of 18 G to 22 G) that causes less pain, but a needle of another G (gauge) is also used at a portion where subcutaneous fat is thick and the sense of pain is relatively small. Note that it is possible to use an injection needle having a thickness other than those under the G standards described above.

As the needle-point shape of the injection needle 2 under general standards, any one of two types is selectively used, that is, a regular bevel (R. B.) and a short bevel (S. B.). The regular bevel has a long diagonally-cut needle point at an angle of 12 degree, and the short bevel has a diagonally-cut needle point shorter than than that of the regular bevel, at an angle of 18 degree. Note that a needle having a needle-point shape other than those two described above can also be used.

Also, a plurality of injection needles having lengths (approximately 3 cm to approximately 20 cm) are prepared, and any one of these is used as the injection needle 2 depending on the portion for use. Specifically, in fine surgery on the corners of the eye or the mouth, or others, a relatively short injection needle having a length of approximately 5 cm is used. In surgery on a wide area such as hip lifting surgery, an injection needle having a length of approximately 15 cm is used.

As depicted in FIG. 2, the implant thread 3 is folded into two at the diagonally-cut tip side of the injection needle 2, with the one end 3b side with a predetermined length being inserted into the pore 2b at the tip of the injection needle 2 and the other end 3a side being held by the fastener 4 along the injection needle 2. Here, the thickness of the implant thread 3 is preferably formed thinner than the thickness of the injection needle 2 because the implant thread 3 is reliably implanted in the affected area of the body. Also, when the implant thread 3 is attached to the injection needle 2 as being folded, the length of two folded portions of the implant thread 3 is preferably shorter than the length of the injection needle 2 so that the implant thread 3 is prevented from getting stuck in the pore 2b of the injection needle 2 can be reliably removed from the fastener 4.

Here, the implant thread 3 for use in the present embodiment is a suture for surgery. As a suture for surgery, any of various threads classified as a combination of (1) a natural-material thread or a synthetic thread, (2) a braided thread (a twisted thread) or a monofilament, and (3) a non-absorbable thread or an absorbable thread can be used. In the acupuncture tool 1 for performing acupuncture in the present embodiment, a thread made of PDS (polydioxanone) having features of (1) a synthetic thread, (2) a filament, and (3) an absorbable thread is used. This PDS (polydioxanone) has a property of being present inside a living body as having a sufficient strength for approximately six months. When the implant thread 3 is implanted in the body, the implant thread 3 is gradually decomposed over one month to six months, and therefore can continuously and repetitively stimulate tissues inside the body near the implant thread 3. As in the present embodiment, if the implant thread 3 is folded into two so that each has an approximately the same length, a resilient force that tries to cause the implant thread 3 to be back to the original linear shape acts in the case of PDS (polydioxanone), which may provide more stimulation.

Here, while the implant thread 3 has the other end 3a side held by the fastener 4, the implant thread 3 can be inserted (implanted) as being held and supported by hand or the like for length adjustment, without using the fastener 4.

The fastener 4 is mounted on the injection needle 2, holding the other end 3b side of the implant thread 3 along the injection needle 2. The fastener 4 holds the implant thread 3 while moving along the injection needle 2 (in a direction indicated in an arrow in FIG. 1) during a period from a time when the injection needle 2 is inserted into the affected area of the body to a time when the implant thread 3 is implanted in the body. As the fastener 4 according to the present embodiment, a cushioning elastic member such as a sponge is used, acting an appropriate pressing force in a range of capable of moving along the injection needle 2. With this, the fastener 4 reliably fastens the implant thread 3, which is made of PDS (polydioxanone) described above and is folded into two to try to be back to the original shape, to the injection needle 2.

Here, while one fastener 4 is used in the present embodiment, a plurality fasteners 4 may be used according to the length of the injection needle 2 and the length of the implant thread 3. Also, the fastener 4 may be used with its color variously changed according to the type of chemical solution or pharmaceutical, the affected area, the lengths of the injection needle 2 and implant thread 3, or others.

The operating unit 5 is integrally provided on the base end 2a side of the injection needle 2. When the injection needle 2 is inserted into and extracted from the body, the operating unit 5 is operated as being held by hand. At this time, since the operating unit 5 is integrally provided to the injection needle 2, the operating unit 5 never comes off. The operating unit 5 has a step part 5a, and is configured in a manner such that this step part 5a and a cap 6 (which will be described further below) fit in each other. The operating unit 5 is made by using any of various materials. For example, if the operating unit 5 is made of resin, the operating unit 5 is easily molded integrally with the injection needle 2, and also can be made light weight.

As depicted in FIG. 3, the cap 6 is cylindrical, with its one end side (an operating unit 5 side) open and the other end side (a tip side of the injection needle 2) closed, and is placed to cover the injection needle 2 and the implant thread 3 and fit in the step part 5a of the operating unit 5. The cap 6 is also placed so as not to be in contact with the injection needle 2 and the fastener 4. With the cap 6 being placed, an external contact with the injection needle 2, the implant thread 3, and the fastener 4 can be prevented, and cleanliness can be kept. In the case of the implant thread 3 having a chemical solution or pharmaceutical, the effect of that chemical solution or pharmaceutical can be maintained long.

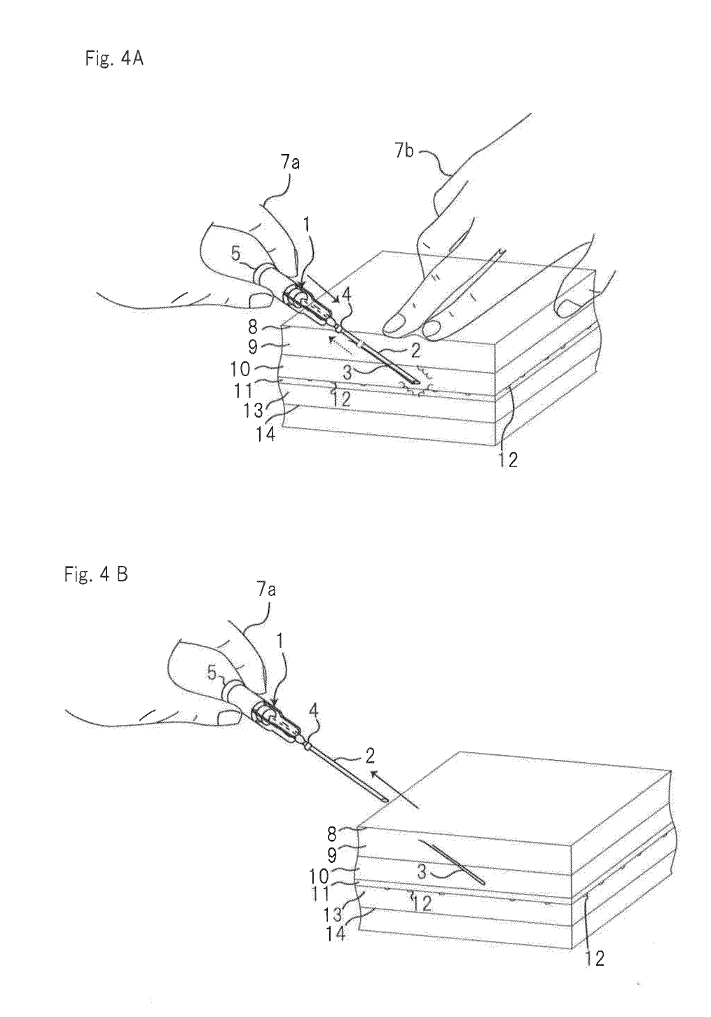

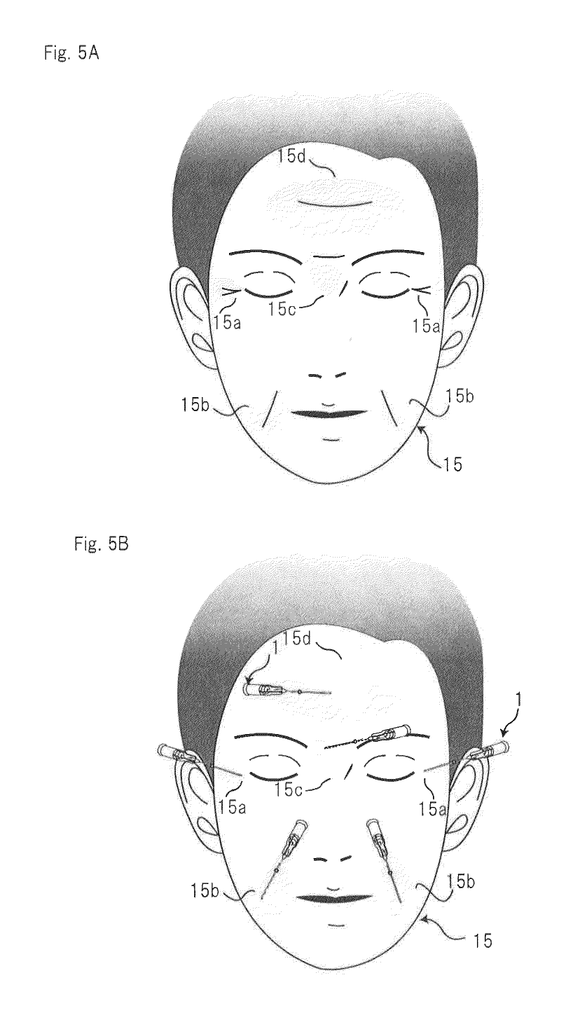

Next, examples of use of the acupuncture tool 1 for performing acupuncture in the present embodiment are described by using the drawings. FIG. 4A and FIG. 4B are diagrams for describing the state in which the implant thread 3 is implanted by the injection needle 2 of FIG. 1 in the inside of the skin, FIG. 4A depicting that the tip is inserted into the inside of the skin and FIG. 4B depicting that the tip is extracted from the inside of the skin. FIG. 5A and FIG. 5B are diagrams of an example of use of the acupuncture tool 1 of FIG. 1, FIG. 5A depicting parts where wrinkles are formed on the face and FIG. 5B depicting the state of inserting the acupuncture tool.

As depicted in FIG. 4A, with the operating unit 5 being gripped by one hand 7a, the injection needle 2 and the implant thread 3 are inserted into the inside of the skin in a direction indicated by an arrow. At this time, the fastener 4 moves in a direction indicated by an arrow in FIG. 4A. The acupuncture tool 1 for performing acupuncture is applied along a specific meridian and a special acupuncture point and muscle for use in acupuncture. Here, the inner tissue of the skin broadly includes a skin, a lymphatic system, a muscle, and a bone in the descending order from an upper layer of the skin. In more detail, as depicted in FIG. 4A and FIG. 4B, an emidermis 8, a dermis 9, a fibrous septum 10, a fascia 11, a blood vessel 12 placed between the fibrous septum 10 and the fascia 11, a muscle 13, and a motor nerve 14. In this inner tissue of the skin, stimulation is provided with the tip of the injection needle 2 (in FIG. 4A, stimulation is provided to the fibrous septum 10). Simultaneously or immediately thereafter, a portion near the tip of the injection needle 2 is pressed by the other hand 7b from the skin surface.

Subsequently, without an interval, as depicted in FIG. 4B, the injection needle 2 is extracted from the body along the direction of insertion. At this time, the implant thread 3 is left in the affected area of the body (the dermis 9 and the fibrous septum 10 in FIG. 4B). As such, when the injection needle 2 is inserted into the affected area of the body, the implant thread 3 is also simultaneously implanted in the body, and then only the injection needle 2 is extracted with the implant thread 3 left in the body. Then, during a period from a time when the injection needle 2 is inserted into the affected area of the body to a time when the implant thread 3 is implanted in the body, the fastener 4 holds the implant thread 3 while moving along the injection needle 2. Therefore, the implant thread 3 can be reliably implanted in the body.

As described above, with the implant thread 3 implanted in the body, the respiratory muscle in the body is adjusted to let the blood vessel 12 communicate, the balance between vertical and lateral directions of the body is corrected, and their ratio and symmetry are adjusted. As a result, the aged dermis layer and a sub-muscular aponeurotic system (SMAS) that surround an acupuncture point receive stimulation to strengthen the muscle, thereby generating collagen. Through this process, an effect of reducing the sag of the skin (lifting) can be obtained with wrinkles being removed and the skin tissue being stabilized. Also, with addition of, for example, stimulation causing the implant thread 3 made of PDS (polydioxanone) and folded into two directions to try to be back to the original linear shape, the muscle is back to a correct position, and the muscles of the bust and the hip become elastic while developing.

For example, as depicted in FIG. 5A, eye corner areas 15a, mouth corner areas 15b, a glabella area 15c and a forehead area 15d in a face 15 undergo acupuncture. As depicted in FIG. 5B, the acupuncture tool 1 is inserted along a wrinkle formed in each area. At this time, for example, when a plurality of wrinkles are present as in the eye corner area 15a, the acupuncture methodology is variously changed in a manner such that acupuncture with the acupuncture tool 1 is performed with a number of times as many as the number of wrinkles or more effective parts undergo acupuncture with a number of times less than the number of winkles.

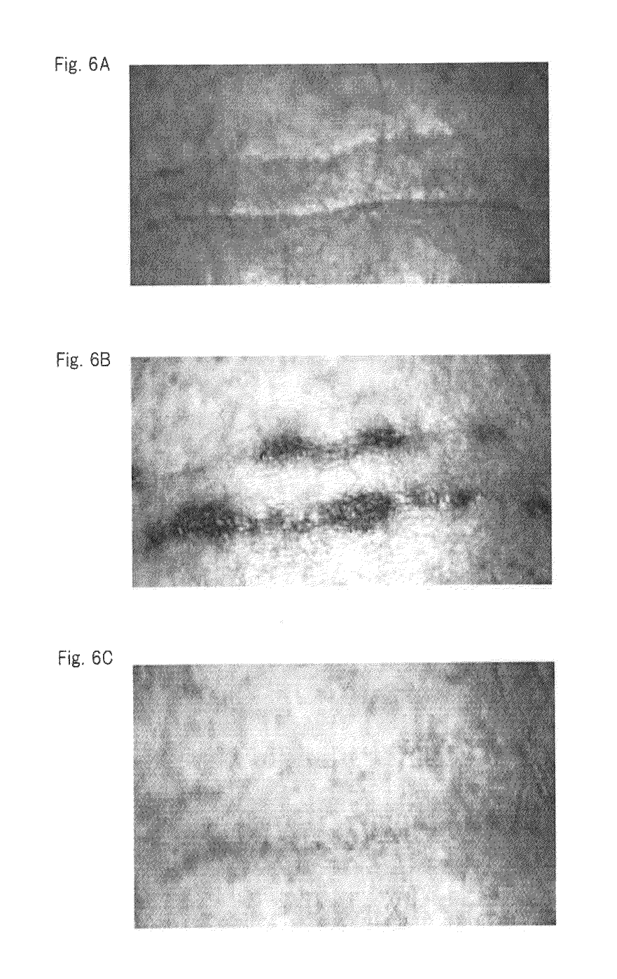

Next, an actual example is used to describe the result of inserting the acupuncture tool 1 for performing acupuncture of the present invention into the face 15. FIG. 6A to FIG. 6C are diagrams for describing the progress of effects of the acupuncture tool 1 for performing acupuncture of the present invention on wrinkles on the forehead, FIG. 6A depicting the state before the use of the acupuncture tool 1, FIG. 6B depicting a first stage after the use of the acupuncture tool 1, and FIG. 6C depicting the final stage after the use of the acupuncture tool 1.

As depicted in FIG. 6A, two wrinkles are clearly formed on the forehead. When the acupuncture tool 1 for performing acupuncture of the present invention is inserted into a portion near these wrinkles, firstly as a first stage, as depicted in FIG. 6B, thick blotches (or a kind of blotches) become standing out. Then, these thick blotches (or the kind of blotches) become disappearing and, eventually, disappear completely as depicted in FIG. 6C.

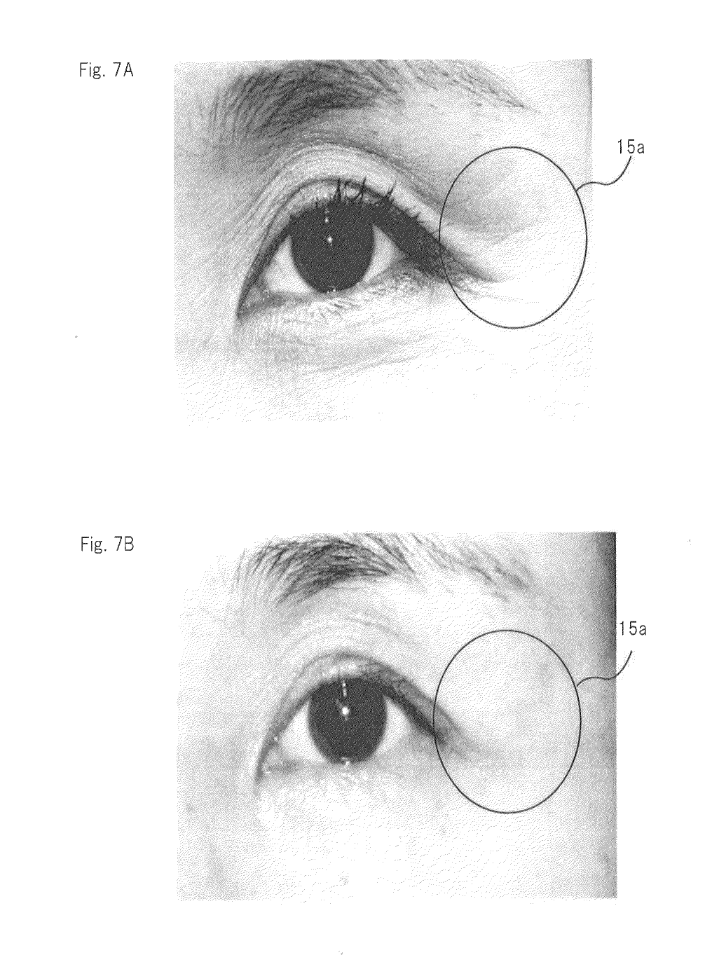

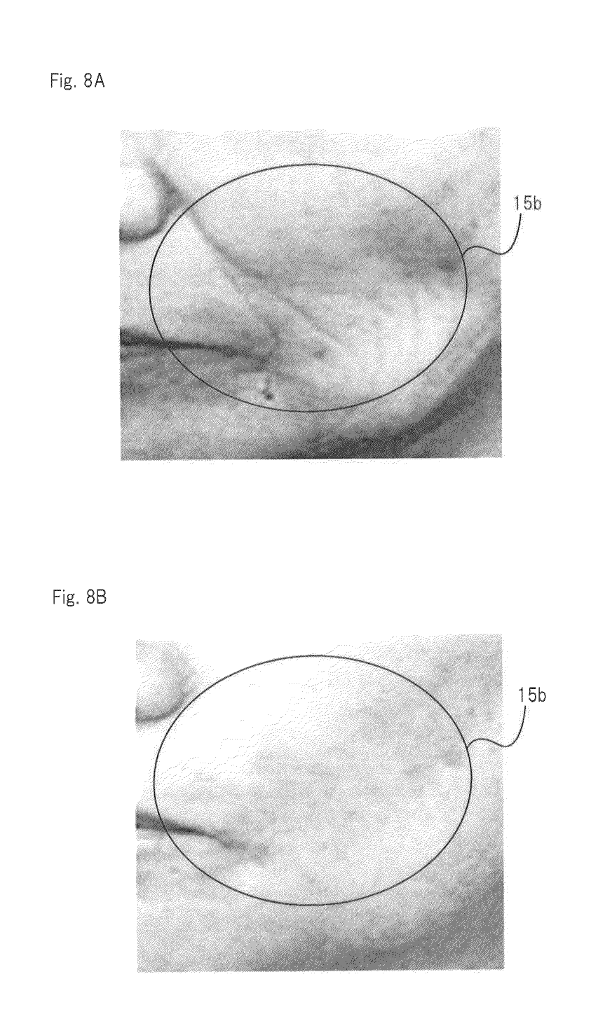

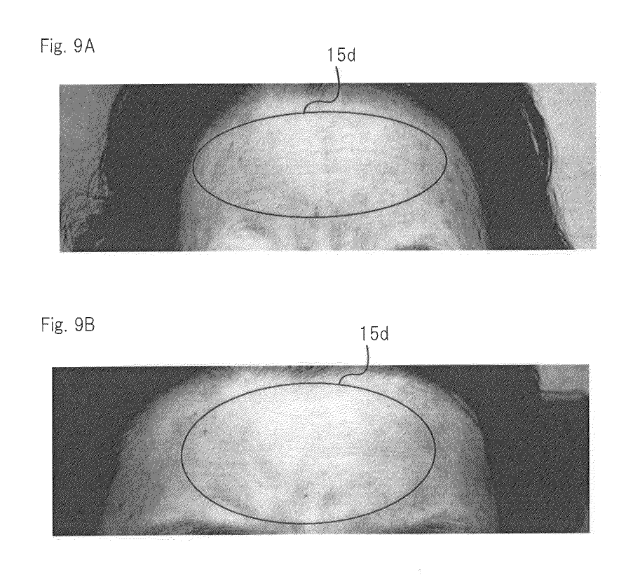

Regarding the blotches in each of the areas 15a, 15b, and 15c, and 15d on the face 15 disappearing through the process described above, effects are specifically described. FIG. 7A and. FIG. 7B are diagrams for describing effects of the acupuncture tool 1 for performing acupuncture of the present invention on wrinkles at the corners of the eyes, FIG. 7A depicting the state before the use of the acupuncture tool 1 and FIG. 7B depicting the state after the use of the acupuncture tool 1. FIG. 8A and FIG. 8B are diagrams for describing effects of the acupuncture tool 1 for performing acupuncture of the present invention on wrinkles at the corners of the mouth, FIG. 8A depicting the state before the use of the acupuncture tool 1 and FIG. 8B depicting the state after the use of the acupuncture tool 1. FIG. 9A and FIG. 9B are diagrams for describing effects of the acupuncture tool 1 for performing acupuncture of the present invention on wrinkles on the forehead, FIG. 9A depicting the state before the use of the acupuncture tool 1 and FIG. 9B depicting the state after the use of the acupuncture tool 1.

As depicted in FIG. 7A, with the acupuncture tool 1 for performing acupuncture of the present invention being inserted into the eye corner area 15a, it can be clearly found as depicted in FIG. 7B that a plurality of wrinkles formed in the eye corner area 15a completely disappear and the skin becomes taut. Also as depicted in FIG. 8A, with the acupuncture tool 1 for performing acupuncture of the present invention being inserted into the mouth corner area 15b, it can be clearly found as depicted in FIG. 8B that a large wrinkle like a slanted line at a corner of the mouth clearly formed in the mouth corner area 15b completely disappears. Furthermore, as depicted in FIG. 9A, with the acupuncture tool 1 for performing acupuncture of the present invention being inserted into the forehead area 15d, it can be clearly found as depicted in FIG. 9B that a plurality of wrinkles clearly formed in the forehead area 15d and laterally extending disappear almost completely.

The wrinkle removal methodology described above can be effective not only on wrinkles formed on the face but also on those formed on the neck.

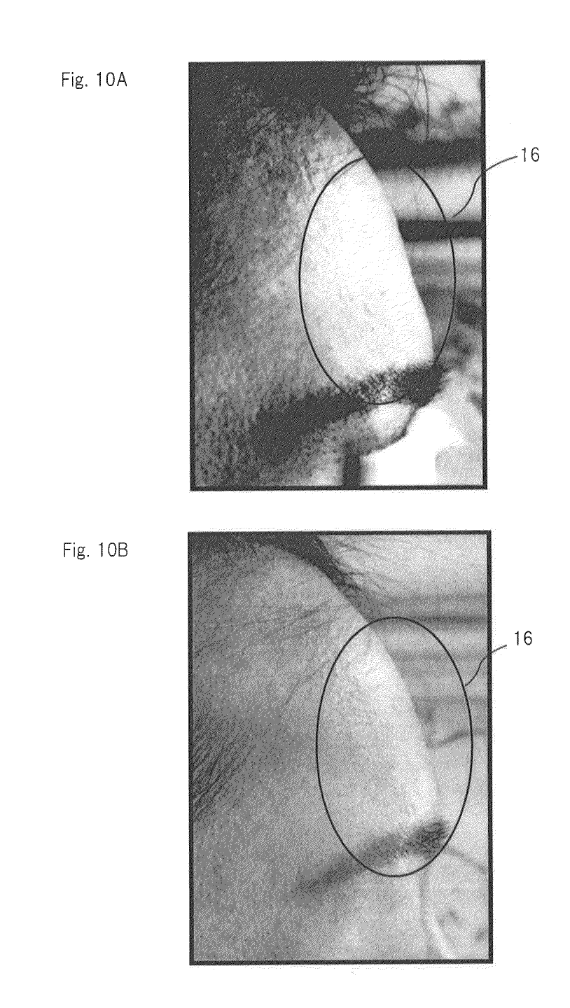

Next, the results of performing acupuncture with the acupuncture tool 1 for performing acupuncture of the present invention on body parts other than the face 15 are described by using actual examples. FIG. 10A and FIG. 10B are diagrams for describing effects of the acupuncture tool 1 for performing acupuncture of the present invention on surgery for the forehead, FIG. 10A depicting the state before the use of the acupuncture tool 1 and FIG. 10B depicting the state after the use of the acupuncture tool 1.

As depicted in FIG. 10A, the forehead is approximately flat (is bowed inward a little). With the acupuncture tool 1 for performing acupuncture of the present invention being inserted into the forehead, it can be found as depicted in FIG. 10B that the forehead is swelled and has some height. As such, only with the acupuncture tool 1 for performing acupuncture of the present invention being inserted into the forehead without performing incision, the forehead can be swelled.

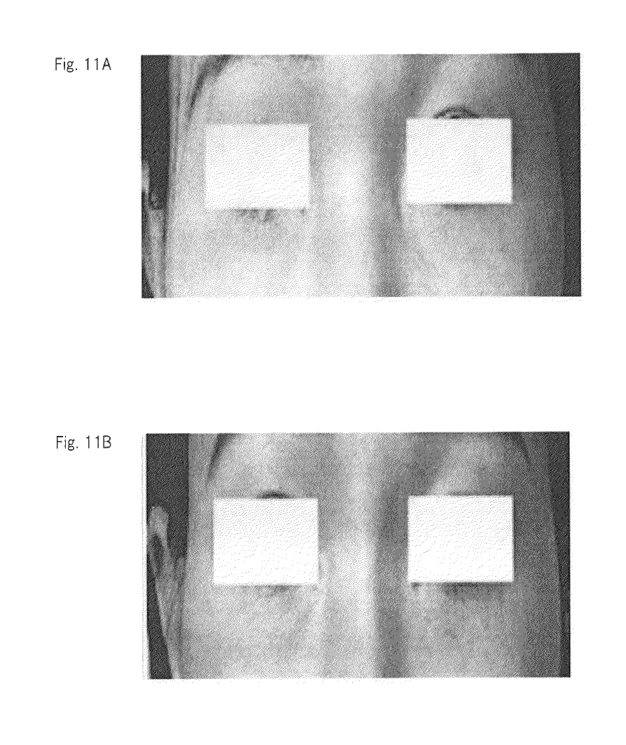

FIG. 11A and FIG. 11B are diagrams for describing effects of the acupuncture tool 1 for performing acupuncture of the present invention on surgery for straightening the nasal muscle, FIG. 11A depicting the state before the use of the acupuncture tool 1 and FIG. 11B depicting the state after the use of the acupuncture tool 1.

As depicted in FIG. 11A, the nasal muscle is not so well-formed. With the acupuncture tool 1 for performing acupuncture of the present invention being inserted into a muscle around the nose, it can be found as depicted in FIG. 11B that the nasal muscle is well-formed and has some height. As such, only with the acupuncture tool 1 for performing acupuncture of the present invention being inserted into the nose without performing incision, an effect of changing the poorly-formed nose into the one having some height can be obtained. In addition, wrinkles on the glabella and wrinkles like slanted lines at the corners of the mouth can be removed, and symptoms such as rhinitis can also be alleviated.

The nasal muscle straightening methodology described above is also effective for straightening the hawk nose. With the acupuncture tool 1 for performing acupuncture of the present invention being inserted, the hawk nose can be straightened to become a nose with a liner nasal muscle.

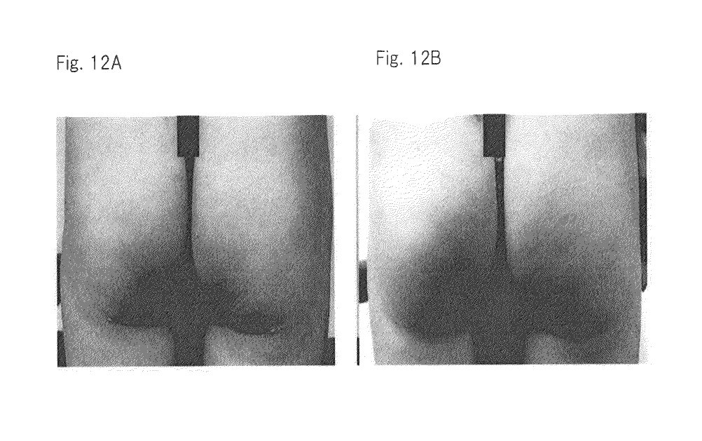

FIG. 12A and FIG. 12B are diagrams for describing effects of the acupuncture tool 1 for performing acupuncture of the present invention in reducing the sag of the buttocks (lifting the hips), FIG. 12A depicting the state before the use of the acupuncture tool 1 and FIG. 12B depicting the state after the use of the acupuncture tool 1.

With the acupuncture tool 1 for performing acupuncture of the present invention being inserted into the buttocks, the sag of the buttocks as depicted in FIG. 12A is reduced (the hips are lifted) as depicted in FIG. 12B. The muscles required to form non-saggy beautiful buttocks are muscle tissues of several types including the gluteus maximus muscle and the gluteus minimus muscle. With the acupuncture tool 1 for performing acupuncture of the present invention being inserted, elasticity of these muscles are recovered, and a radical cure for correcting the balance between the pelvic bone and the vertebrae is performed. Also, unbalanced hips can be corrected. Furthermore, with the effect of acupuncture and the implant thread 3, a hip pain can be treated, menstrual irregularity can be reduced, the functions of the large intestines can be activated, and indigestion can also be reduced.

As with the hip lifting described above, the sag of the breasts can also be reduced with the acupuncture tool 1 for performing acupuncture of the present invention, thereby obtaining an effect of making non-sagged breasts (so-called lifting the breasts).

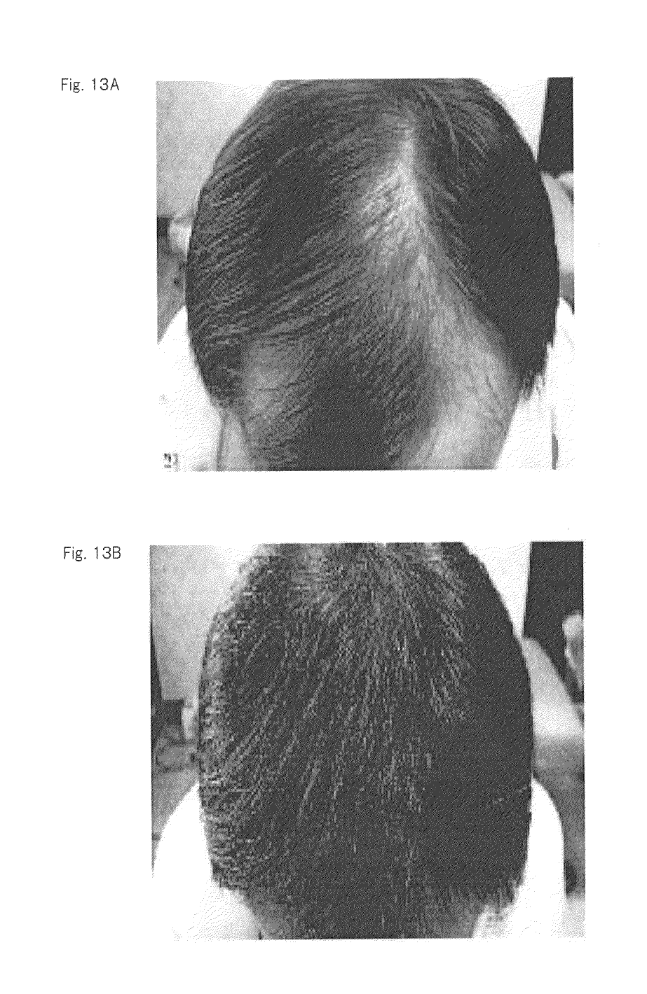

FIG. 13A and FIG. 13B are diagrams for describing effects of the acupuncture tool 1 for performing acupuncture of the present invention in a treatment for increasing hairs, FIG. 13A depicting the state before the use of the acupuncture tool 1 and FIG. 13B depicting the state after the use of the acupuncture tool 1.

In the case of a partially-exposed scalp with thin (less) hairs as depicted in FIG. 13A, the acupuncture tool 1 for performing acupuncture of the present invention is inserted into the affected area on the scalp. As a result, it can be found as depicted in FIG. 13B that hairs are increased to the extent to which the scalp cannot be seen. This is because hair roots are restored with the renewal of cells.

Also, the acupuncture tool 1 for performing acupuncture of the present invention can also be applied to a prominence and dent of the cheek bone. In this case, in the conventional cosmetic surgery, an operation with many side effects is performed, such as Botox or silicon injecting operation. With the acupuncture tool 1 for performing acupuncture of the present invention, the prominence and dent of the cheek bone can be solved, smoothing the contour of the face well. By performing the operation using the acupuncture tool 1 in consideration, with highest priority, of the golden ratio of the face, that is, a ratio of approximately 1:16 human thinks intrinsically beautiful, the most balanced shape suitable for the contour of the face and the shape of each part of the individual is created from the healing power of the body, and the muscles and skeleton are recovered to the ideal model. As a result, the protruding cheek bone is reduced, and a dented cheek flesh part is swelled, thereby creating entire facial beauty with balanced parts.

Furthermore, the acupuncture tool 1 for performing acupuncture of the present invention can also be applied to the backbone not bilaterally symmetrical. In this case, with the acupuncture tool 1 for performing acupuncture of the present invention being inserted into the entire backbone, the backbone becomes bilaterally symmetrical, and the body can be found as balanced. With the use of the needle and the medical method together, that is, with stimulation of the affected area of the body by using the injection needle 2 and implantation of the implant thread 3, “ki” (or “qi”) is changed (is let breathe), the axis of the body is straightened, and bilateral symmetry of the body and the ratio of respective parts and muscles are correctly restored.

Description has been made by mainly taking the face, the backbone, the buttocks, and others as examples of the affected area of the body to which the acupuncture tool 1 is to be applied in the present embodiment described above. However, the shape of the acupuncture tool 1, specifically, the lengths of the injection needle 2 and the implant thread 3, can be variously changed, the acupuncture tool 1 can be applied to a wide range of affected areas of the body and, for example, can be applied to the knee, the abdomen, or others.

Also, in the conventional acupuncture treatment, the acupuncture tool is required to be in the state of being inserted for a while (approximately twenty minutes to approximately thirty minutes). By contrast, in the acupuncture tool 1 for performing acupuncture of the present invention, the implant thread 3 serves as an acupuncture tool, and no needle is required to be in the state of being inserted. This does not cause physical pain or fatigue. Furthermore, since stimulation like the one from the acupuncture tool is continuously provided until the implant thread 3 is completely melted, the effect is higher as ever. A high effect can be obtained particularly in treating shoulder stiffness. Still further, in the acupuncture tool 1 of the present invention, a high effect equivalent to or higher than the conventional effect can be obtained with the number of acupuncture tools smaller than ever.

Still further, in the present embodiment, PDS (polydioxanone) is used for the implant thread 3. With this, the implant thread 3 is decomposed approximately one to six months after being implanted in the affected area of the body. Even after decomposition, the healing power is supported, and the effects on elasticity of the skin and the pain of the affected area continue. Thereafter, the implant thread 3 is changed into a part of the body, and does not have a side effect on the body. For example, if the cells in the affected area of the body are renewed back to ten years, the cells become older from the state renewed back to ten years. Therefore, by implanting the implant thread 3 one year thereafter, the elastic state of the skin can be maintained.

What is claimed is:

1. An acupuncture tool for performing acupuncture to treat a disease, promote health, and carry out other purposes by stimulating a body, the acupuncture tool comprising:
an injection needle; and
an implant thread to be implanted in the body,
the implant thread having one end side being inserted into a pore at a tip side of the injection needle and another end side being drawn so that when the injection needle is inserted into an affected area of the body, the implant thread is also simultaneously implanted in the body and the injection needle is then extracted with the implant thread left in the body, and
the injection needle having a predetermined fastener movably mounted thereon, the fastener holding the other end side of the implant thread along the injection needle, and the fastener configured to move in a direction reverse to a direction of inserting the injection needle while the fastener is holding the implant thread during a period from a time when the injection needle is inserted into the affected area of the body to a time when the implant thread is implanted in the body.

2. The acupuncture tool for performing acupuncture according to claim 1, wherein
the fastener is circular along an outer perimeter of the injection needle, and is made by using a cushioning elastic member.

3. The acupuncture tool for performing acupuncture according to claim 1, wherein
a drawn length of the implant thread to be drawn from the pore of the injection needle is set longer than an insertion length of the implant thread to be inserted into the pore of the injection needle.

4. The acupuncture tool for performing acupuncture according to claim 2, wherein
a drawn length of the implant thread to be drawn from the pore of the injection needle is set longer than an insertion length of the implant thread to be inserted into the pore of the injection needle.

5. The acupuncture tool for performing acupuncture according to claim 1, wherein
a base end side of the injection needle and a resin-made operating unit are integrally provided, and a cap covering the injection needle and the implant thread is configured to removably fit in the operating unit, and
the injection needle and the fastener are covered so as not to be in contact with the cap.

6. The acupuncture tool for performing acupuncture according to claim 2, wherein
a base end side of the injection needle and a resin-made operating unit are integrally provided, and a cap covering the injection needle and the implant thread is configured to removably fit in the operating unit, and
the injection needle and the fastener are covered so as not to be in contact with the cap.

7. The acupuncture tool for performing acupuncture according to claim 3, wherein
a base end side of the injection needle and a resin-made operating unit are integrally provided, and a cap covering the injection needle and the implant thread is configured to removably fit in the operating unit, and
the injection needle and the fastener are covered so as not to be in contact with the cap.

8. The acupuncture tool for performing acupuncture according to claim 1, wherein
the injection needle is an injection needle for surgery, and the implant thread is a suture for surgery.

9. The acupuncture tool for performing acupuncture according to claim 2, wherein
the injection needle is an injection needle for surgery, and the implant thread is a suture for surgery.

10. The acupuncture tool for performing acupuncture according to claim 3, wherein
the injection needle is an injection needle for surgery, and the implant thread is a suture for surgery.

11. The acupuncture tool for performing acupuncture according to claim 4, wherein
the injection needle is an injection needle for surgery, and the implant thread is a suture for surgery.
